(19) United States
(12) Patent Application Publication
(10) Pub. No.: US 2013/0109895 A1
(43) Pub. Date: May 2, 2013

(54) LOW TEMPERATURE ADSORBENT FOR REMOVING SULFUR FROM FUEL

(71) Applicants: William J. Novak, Bedminster, NJ (US); Joseph E. Gatt, Annandale, NJ (US)

(72) Inventors: William J. Novak, Bedminster, NJ (US); Joseph E. Gatt, Annandale, NJ (US)

(73) Assignee: ExxonMobil Research and Engineering Company, Annandale, NJ (US)

(21) Appl. No.: 13/622,043

(22) Filed: Sep. 18, 2012

Related U.S. Application Data
(60) Provisional application No. 61/538,492, filed on Sep. 23, 2011.

(51) Int. Cl. C07C 7/12; C10G 25/05

(52) U.S. Cl. CPC C07C 7/12; C10G 25/05
USPC 585/240; 208/99; 208/246; 585/823

(57) ABSTRACT

The present invention relates to methods for removing sulfur from a hydrocarbon fuel or fuel precursor feedstream, such as methods comprising contacting a hydrocarbon fuel or fuel precursor feedstream having a relatively low sulfur content with a sulfur sorbent material comprising an active copper component disposed on a zeolitic and/or mesoporous support under conditions sufficient to reduce the sulfur content by at least 20 wt % and/or to about 15 wppm or below, thus forming a hydrocarbon fuel product. In some advantageous embodiments, the contacting conditions can include a temperature of about 392° F. (about 200° C.) or less.

### CROSS-REFERENCE TO RELATED APPLICATIONS

This application claims priority to U.S. Provisional Application Ser. No. 61/538,492 filed Sep. 23, 2011, herein incorporated by reference in its entirety.

### FIELD OF THE INVENTION

This invention provides a method for trimming sulfur from a hydrocarbon fuel or fuel precursor feedstream having an already relatively low sulfur content through use of a sulfur sorbent material. In particular, the sulfur sorbent material can comprise an active copper component disposed on a zeolitic and/or mesoporous support.

### BACKGROUND OF THE INVENTION

Using adsorbent materials as a means of trapping sulfur compounds is a well known means of desulfurization, but is generally less preferred (at least currently) for hydrocarbon feeds than hydrogen-based hydrodesulfurization methods. Both methods are typically performed at relatively high temperatures, but hydrogen-based desulfurization processes can be more easily tuned in severity to deal with the broadest variety of sulfur compounds, from simple sulfur removal to difficult sulfur removal. Furthermore, current sulfur trapping materials/catalysts (such as high nickel, copper on alumina, manganese oxide, and the like) generally have limited sorption capacity, thus requiring frequent changeout in refinery-type settings, and are usually only once-through materials, since their regenerability for reuse is typically not practical. However, hydrogen-based desulfurization processes tend to use expensive catalysts, and the hydrogen gas itself is routinely consumed in such processes and relatively expensive to source in the relatively high purity necessary to effectively remove the most difficult sulfur compounds typically present in fuel-oriented feedstreams, such as mineral crude oils.

There are some prior art references involving the disclosure of sulfur adsorbents based on copper(I) and zeolite Y. For example, U.S. Patent Application Publication No. 2009/0118528 A1 discloses such a sulfur adsorbent material, but for the application of removal of sulfur compounds present specifically in natural gas feedstreams, not from sulfur compounds present specifically in liquid/fuel-based feedstreams. Other references relevant to sulfur removal by sorbent materials include, but are not necessarily limited to, PCT Publication No. WO 03/054117, and U.S. Pat. Nos. 5,248,321, 6,033,461, 6,215,037, 6,423,881, 6,867,166, 6,911,569, 7,029,547, 7,053,256, 7,094,333, and 7,148,389.

The present invention involves adapting copper-based sorbents that can operate at lower temperatures and that can be regenerated for multiple reuses to efficiently remove relatively difficult sulfur compounds from hydrocarbon feedstreams typical of naphtha to diesel boiling range materials. Since such feedstreams, when raw or unprocessed, tend to have relatively large sulfur contents, and since the capacity of adsorbents can generally be limited, it can be advantageous to attain a repeatedly regenerable, low-temperature sorbent process tailored to such feedstreams. In particular, adaptation of sulfur sorbent materials specifically to sulfur trimming methods (or methods for removal of the last little bit of sulfur content) to attain on-spec and/or lower sulfur content hydrocarbon/fuel products can provide an advantage in both cost and resources.

### SUMMARY OF TIT INVENTION

One aspect of the invention relates to a method for removing sulfur from a hydrocarbon fuel or fuel precursor feedstream comprising: contacting a hydrocarbon fuel or fuel precursor feedstream having a relatively low sulfur content (e.g., from about 2 wppm to about 100 wppm) with a sulfur sorbent material comprising an active copper component disposed on a zeolitic and/or mesoporous support (e.g., comprising a Cu(I) component disposed on zeolite Y) under conditions sufficient to reduce the sulfur content by at least about 20 wt % and/or to about 15 wppm or below, thus forming a hydrocarbon fuel product, wherein the conditions include at least a temperature of about 392° F. (about 200° C.) or less.

In one embodiment, the hydrocarbon fuel or fuel precursor feedstream can comprise a naphtha stream, a gasoline precursor stream, a gasoline fuel stream, or a combination thereof, and can also exhibit one or more of the following: an IBP of at least about 90° F. (about 32° C.); an MP of about 150° F. (about 66° C.) or less; a T5 boiling point of at least about 100° F. (about 38° C.); a T5 boiling point of about 200° F. (about 93° C.) or less; a T95 boiling point of at least about 325° F. (about 163° C.); a T95 boiling point of about 425° F. (about 218° C.) or less; an FBP of at least about 350° F. (about 177° C.); and an FBP of about 425° F. (about 218° C.) or less. In such an embodiment, one or more of the following may further be satisfied: the hydrocarbon fuel or fuel precursor feedstream can exhibit a sulfur content from about 10 wppm to about 50 wppm; the sulfur sorbent material can comprise an active copper (1) component disposed on zeolite Y; the contacting conditions can be sufficient to reduce the sulfur content by at least about 20 to 80 wt %; and the contacting conditions can include a temperature of about 302° F. (about 150° C.) or less.

In another embodiment, the hydrocarbon fuel or fuel precursor feedstream can comprise a jet fuel precursor stream, a jet fuel stream, a kero precursor stream, a kero fuel stream, or a combination thereof, and can also exhibit one or more of the following: an IBP of at least about 150° F. (about 66° C.); IBP of about 375° F. (about 191° C.) or less; a T5 boiling point of at least about 200° F. (about 93° C.); a T5 boiling point of about 400° F. (about 204° C.) or less; a T95 boiling point of at least about 500° F. (about 260° C.); T95 boiling point of about 575° F. (about 302° C.) or less; an FBP of at least about 500° F. (about 260° C.); and an FBP of about 625° F. (about 329° C.) or less.

In still another embodiment, the hydrocarbon fuel or fuel precursor feedstream can comprise a diesel precursor stream, a hydrotreated diesel stream, a diesel fuel stream, or a combination thereof, and can also exhibit one or more of the following: an IBP of at least about 350° F. (about 177° C.); an IBP of about 450° F. (about 232° C.) or less; a T5 boiling point of at least about 375° F. (about 191° C.); a T5 boiling point of about 450° F. (about 231° C.) or less; a T95 boiling point of at least about 650° F. (about 343° C.); a T95 boiling point of about 725° F. (about 385° C.) or less; an FBP of at least about 675° F. (about 357° C.); and an FBP of about 750° F. (about 399° C.) or less. In such an embodiment, one or more of the following may further be satisfied: the hydrocarbon fuel or fuel precursor feedstream can exhibit a sulfur content from about 15 wppm to about 100 wppm; the sulfur sorbent material can comprise an active copper (I) component disposed on zeolite Y; the contacting conditions can be sufficient to reduce the sulfur content by at least about 30 to 90 wt %; and the contacting conditions can include a temperature of about 149° F. (about 65° C.) or less.

### BRIEF DESCRIPTION OF THE FIGURES

FIG. 1 shows a plot of sulfur product content versus normalized cumulative feed volume for a method of removing sulfur from a naphtha boiling range feedstream according to the invention.

FIG. 2 shows a plot of sulfur product content versus cumulative feed volume/rate for a method of removing sulfur from a diesel boiling range feedstream according to the invention.

### DETAILED DESCRIPTION OF THE EMBODIMENTS

in one aspect, the present invention relates to a method for removing sulfur from a hydrocarbon fuel or fuel precursor feedstream comprising: contacting a hydrocarbon fuel or fuel precursor feedstream having an already relatively low sulfur content with a sulfur sorbent material comprising an active copper component disposed on a zeolitic and/or mesoporous support under conditions sufficient to reduce the sulfur content, thus forming a hydrocarbon fuel product, wherein the conditions include at least a temperature of about 392° F. (about 200° C.) or less and optionally a pressure at which the hydrocarbon fuel or fuel precursor feedstream remains substantially liquid.

The hydrocarbon fuel or fuel precursor feedstream can comprise, consist essentially of, or consist of a naphtha stream, a gasoline precursor stream, a gasoline fuel stream, a diesel precursor stream, a hydrotreated diesel stream, a diesel fuel stream, a jet fuel precursor stream, a jet fuel stream, a kero precursor stream, a kero fuel stream, or the like, or a combination thereof. Additionally or alternately, as a result, the hydrocarbon fuel product can comprise, consist essentially of, or consist of a gasoline fuel, a jet fuel, a kerosene fuel, a diesel fuel, or the like, or a combination thereof.

Another way to characterize the hydrocarbon fuel or fuel precursor feedstream used in methods according to the invention can be based on its origin, namely as a mineral hydrocarbon feedstream, a biocomponent hydrocarbon feedstream, or a mixture thereof. A mineral hydrocarbon feedstream refers to a hydrocarbon feedstream derived from the earth (e.g., crude oil, oil shale, etc.) that has optionally been subjected to one or more separation and/or other refining processes. The mineral hydrocarbon feedstream can be a petroleum feedstream, e.g., boiling in the naphtha to diesel range. Based on the relatively low sulfur specification on the feedstream, suitable mineral feeds can have previously undergone hydrotreatment, hydrocracking, fluid catalytic cracking, steam cracking, hydroisomerization/hydrodewaxing, aromatic saturation, some other form of hydroprocessing, or a combination thereof.

In the discussion below, biocomponent feedstock refers to a hydrocarbon feedstock derived from a biological raw material component, from biocomponent sources such as vegetable, animal, fish, and/or algae. Note that, for the purposes of this document, vegetable fats/oils refer generally to any plant based material, and can include fat/oils derived from a source such as plants of the genus Jatropha. Generally, the biocomponent sources can include vegetable fats/oils, animal fats/oils, fish oils, pyrolysis oils, and algae lipids/oils, as well as components of such materials, and in some embodiments can specifically include one or more type of lipid compounds. Lipid compounds are typically biological compounds that are insoluble in water, but soluble in nonpolar (or fat) solvents. Non-limiting examples of such solvents include alcohols, ethers, chloroform, alkyl acetates, benzene, and combinations thereof.

Major classes of lipids include, but are not necessarily limited to, fatty acids, glycerol-derived lipids (including fats, oils and phospholipids), sphingosine-derived lipids (including ceramides, cerebrosides, gangliosides, and sphingomyelins), steroids and their derivatives, terpenes and their derivatives, fat-soluble vitamins, certain aromatic compounds, and long-chain alcohols and waxes.

in living organisms, lipids generally serve as the basis for cell membranes and as a form of fuel storage. Lipids can also be found conjugated with proteins or carbohydrates, such as in the form of lipoproteins and lipopolysaccharides.

Examples of vegetable oils that can be raised in accordance with this invention include, but are not limited to rapeseed (canola) oil, soybean oil, coconut oil, sunflower oil, palm oil, palm kernel oil, peanut oil, linseed oil, tall oil, corn oil, castor oil, jatropha oil, jojoba oil, olive oil, flaxseed oil, camelina oil, safflower oil, babassu oil, tallow oil, and rice bran oil.

Vegetable oils as referred to herein can also include processed vegetable oil material. Non-limiting examples of processed vegetable oil material include fatty acids and fatty acid alkyl esters. Alkyl esters typically include C₁-C₅ alkyl esters. One or more of methyl, ethyl, and propyl esters are preferred.

Examples of animal fats that can be used in accordance with the invention include, but are not limited to, beef fat (tallow), hog fat (lard), turkey fat, fish fat/oil, and chicken fat. The animal fats can be obtained from any suitable source including restaurants and meat production facilities.

Animal fats as referred to herein also include processed animal fat material. Non-limiting examples of processed animal fat material include fatty acids and fatty acid alkyl esters, Alkyl esters typically include C₁-C₅ alkyl esters. One or more of methyl, ethyl, and propyl esters are preferred.

Algae oils or lipids are typically contained in algae in the form of membrane components, storage products, and metabolites. Certain algal strains, particularly microalgae such as diatoms and cyanobacteria, contain proportionally high levels of lipids. Algal sources for the algae oils can contain varying amounts, e.g., from 2 wt % to 40 wt % of lipids, based on total weight of the biomass itself.

Algal sources for algae oils include, but are not limited to, unicellular and multicellular algae. Examples of such algae include a rhodophyte, chlorophyte, heterokontophyte, tribophyte, glaucophyte, chlorarachniophyte, euglenoid, haptophyte, cryptomonad, dinoflagellum, phytoplankton, and the like, and combinations thereof. In one embodiment, algae can be of the classes Chlorophyceae and/or Haptophyta. Specific species can include, but are not limited to, Neochloris oleoabundans, Scenedesmus dimorphus, Euglena gracilis, Phaeodactylum tricornutum, Pleurochrysis carterae, Prymnesium parvum, Tetraselmis chui, and Chlamydomonas reinhardtii.

Additionally or alternately, examples of microalgae can include, for example, Achnanthes, Amphiprora, Amphora, Ankistrodesmus, Asteromonas, Boekelovia, Borodinella, Botryococcus, Bracteococcus, Chaetoceros, Carterfa, Chlamydomonas, Chlorococcum, Chlorogonlum, Chlorella, Chroomonas, Chlysosphaera, Cricosphaera, Crypthecodinium, Cryptomonas, Cyclotella, Dunaliella, Ellipsoidon, Emiliania, Eremosphaera, Ernodesmius, Euglena, Franceia, Fragilaria, Gloeothamnion, Haematococcus, Halocafeteria, Hymenomonas, Isochrysis, Lepocinclis, Micractinium, Monoraphidium, Nannochloris, Nannochloropsis, Navicula, Neochloris, Nephrochloris, Nephroselmis, Nitzschia, Ochromonas, Oedogonium, Oocystis, Ostreococcus, Pavlova, Parachlorella, Pascheria, Phaeodactylum, Phagus, Platymonas, Pleurochrysis, Pleurococcus, Prototheca, Pseudochlorella, Pyramimonas, Pyrobotrys, Scenedesmus, Skeletonema, Spyrogyra, Stichococcus, Tetraselmis, Thalassiosira, Viridiella, and Volvex species; including freshwater and marine microalgal species of these or other genera.

Further additionally or alternately, the algae used according to the invention can be characterized as cyanobacteria. Non-limiting examples of cyanobacteria can include, for example, Agmenellum, Anabaena, Anabaenopsis, Anacystis, Aphanizomenon, Arthrospira, Asterocapsa, Borzia, Calothrix, Chamaesiphon, Chlorogloeopsis, Chroococcidiopsis, Chroococcus, Crinalium, Cyanobacterium, Cyanobium, Cyanocystis, Cyanospira, Cyanothece, Cylindrospermopsis, Cylindrospermum, Dactylococcopsis, Dermocarpella, Fischerella, Fremyella, Geitleria, Geitlerinema, Gloeobacter, Giococapsa, Gloeothece, Halospirulina, lyengariella, Leptolyngbya, Limnothrix, Lyngbya, Microcoleus, Microcystis, Myxosarcina, Nodularia, Nostoc, Nostochopsis, Oscillatoria, Phormidium, Planktothrix, Pleurocapsa, Prochlorococcus, Prochloron, Prochlorothrix, Pseudanabaena, Rivularia, Schizothrix, Scytonema, Spirulina, Stanieria, Starria, Stigonema, Symploca, Synechococcus, Synechocystis, Tolypothrix, Trichodesmium, Tychonema, and Xenococcus species, including freshwater and marine cyanobacterial species of these or other genera.

The boiling range of a suitable hydrocarbon fuel or fuel precursor feedstream can be characterized in various manners. One option can be to characterize the amount of the feedstream that boils above about 350° F. (177° C.). At least about 60 wt %, for example at least about 80 wt % or at least about 90 wt %, of a feedstream can boil above about 350° F. (about 177° C.). Additionally or alternately, at least about 60 wt %, for example at least about 80 wt % or at least about 90 wt %, of the feedstream can boil above about 400° F. (about 204° C.). Another option can be to characterize the amount of feed that boils below a temperature value. In addition to, or as an alternative to, the boiling range features described above, at least about 60 wt %, for example at least about 80 wt % or at least about 90 wt %, of a feedstream can boil below about 650° F. (about 343° C.). Further additionally or alternately, at least about 60 wt %, for example at least about 80 wt % or at least about 90 wt %, of a feedstream can boil below about 700° F. (about 371° C.). Still further additionally or alternatively, a feedstock can have a final boiling point of about 825° F. (about 441° C.) or less, for example about 800° F. (about 427° C.) or less, about 750° F. (about 399° C.) or less, or about 700° F. (about 371° C.) or less. Temperature values can be based on ASTM D2887 and/or ASTM D86.

In an embodiment, the hydrocarbon fuel or fuel precursor feedstream can exhibit one or more of the following: an IBP of at least about 90° F. (about 32° C.), thr example at least about 100° F. (about 38° C.), at least about 125° F. (about 52° C.), at least about 150° F. (about 66° C.), at least about 200° F. (about 93° C.) at least about 250° F. (about 121° C.), at least about 300° F. (about 149° C.), at least about 325° F. (about 163° C.), at least about 350° F. (about 177° C.), at least about 375° F. (about 191° C.), at least about 400° F. (about 204° C.), or at least about 425° F. (about 218° C.); an IBP of about 450° F. (about 232° C.) or less, for example about 425° F. (about 218° C.) or less, about 400° F. (about 204° C.) or less, about 375° F. (about 191° C.) or less, about 350° F. (about 177° C.) or less, about 325° F. (about 163° C.) or less, about 300° F. (about 149° C.) or less, about 250° F. (about 121° C.) or less, about 200° F. (about 93° C.) or less, about 150° F. (about 66° C.) or less, about 125° F. (about 52° C.) or less, or about 100° F. (about 38° C.) or less; a T5 boiling point of at least about 100° F. (about 38° C.), for example at least about 110° F. (about 43° C.), at least about 125° F. (about 52° C.), at least about 150° F. (about 66° C.), at least about 200° F. (about 93° C.), at least about 250° F. (about 121° C.), at least about 300° F. (about 149° C.), at least about 325° F. (about 163° C.), at least about 350° F. (about 177° C.), at least about 375° F. (about 191° C.), at least about 400° F. (about 204° C.), at least about 425° F. (about 218° C.), or at least about 450° F. (about 232° C.); a T5 boiling point of about 450° F. (about 232° C.) or less, for example about 425° F. (about 218° C.) or less, about to 400° F. (about 204° C.) or less, about 375° F. (about 191° C.) or less, about 350° F. (about 177° C.) or less, about 325° F. (about 163° C.) or less, about 300° F. (about 149° C.) or less, about 250° F. (about 121° C.) or less, about 200° F. (about 93° C.) or less, about 150° F. (about 66° C.) or less, about 125° F. (about 52° C.) or less, or about 100° F. (about 38° C.) or less; a T95 boiling point of at least about 325° F. (about 163° C.), for example at least at least about 350° F. (about 177° C.), about 375° F. (about 191° C.), at least about 400° F. (about 204° C.), at least about 425° F. (about 218° C.), or at least about 500° F. (about 260° C.), at least about 525° F. (about 274° C.), at least about 550° F. (about 288° C.), at least about 575° F. (about 302° C.), at least about 625° F. (about 329° C.), at least about 650° F. (about 343° C.), at least about 675° F. (about 357° C.), at least about 700° F. (about 371° C.), or at least about 725° F. (about 385° C.); a T95 boiling point of about 725° F. (about 385° C.) or less, for example about 700° F. (about 371° C.) or less, about 675° F. (about 357° C.) or less, about 650° F. (about 343° C.) or less, about 625° F. (about 329° C.) or less, about 575° F. (about 302° C.) or less, about 550° F. (about 288° C.) or less, about 525° F. (about 274° C.) or less, about 500° F. (about 260° C.) or less, about 425° F. (about 218° C.) or less, about 400° F. (about 204° C.) or less, about 375° F. (about 191° C.) or less, or about 350° F. (about 177° C.) or less; an FBP of at least about 350° F. (about 177° C.), for example at least about 375° F. (about 191° C.), at least about 400° F. (about 204° C.), at least about 425° F. (about 218° C.), or at least about 500° F. (about 260° C.), at least about 525° F. (about 274° C.), at least about 550° F. (about 288° C.), at least about 575° F. (about 302° C.), at least about 625° F. (about 329° C.), at least about 650° F. (about 343° C.), at least about 675° F. (about 357° C.), at least about 700° F. (about 371° C.), or at least about 725° F. (about 385° C.) and an FBP of about 750° F. (about 399° C.) or less, for example about 725° F. (about 385° C.) or less, about 700° F. (about 371° C.) or less, about 675° F. (about 357° C.) or less, about 650° F. (about 343° C.) or less, about 625° F. (about 329° C.) or less, about 575° F. (about 302° C.) or less, about 550° F. (about 288° C.) or less, about 525° F. (about 274° C.) or less, about 500° F. (about 260° C.) or less, about 425° F. (about 218° C.) or less, about 400° F. (about 204° C.) or less, about 375° F. (about 191° C.) or less, or about 350° F. (about 177° C.) or less.

Biocomponent based feedstreams according to the invention typically have, and/or can be pre-treated to attain, e.g., relatively low nitrogen and sulfur contents. Instead of nitrogen and/or sulfur, the primary heteroatom component in biocomponent feeds is typically oxygen (although some biocomponent based feeds can exhibit relatively high nitrogen content). Untreated biocomponent diesel boiling range feedstreams, e.g., can include an oxygen content (i.e., a content of oxygen-containing compounds) up to about 10 wt %, for example up to about 12 wt % or up to about 14 wt %. Suitable biocomponent diesel boiling range feedstreams for use according to the methods of the invention, can be hydrotreated to attain a relatively low oxygen content (and/or nitrogen content, similar to the nitrogen contents listed herein), for example about 1 wt % oxygen or less, about 0.5 wt % oxygen or less, about 0.3 wt % oxygen or less, about 0.1 wt % oxygen or less, about 500 wppm oxygen or less, about 300 wppm oxygen or less, about 200 wppm oxygen or less, about 100 wppm oxygen or less, about 75 wppm oxygen or less, about 50 wppm oxygen or less, about 35 wppm oxygen or less, about 30 wppm oxygen or less, about 25 wppm oxygen or less, about 20 wppm oxygen or less, about 15 wppm oxygen or less, about 10 wppm oxygen or less, or about 5 wppm oxygen or less.

In most embodiments, the hydrocarbon fuel or fuel precursor feedstream comprises only mineral hydrocarbon source(s). However, in some embodiments, the hydrocarbon fuel or fuel precursor feedstream can include some portion of a feed having biocomponent (renewable) origin, which can include or be a hydrotreated vegetable oil feed, a hydrotreated fatty acid alkyl ester feed, or another type of hydrotreated biocomponent feed. A hydrotreated biocomponent feed can be a biocomponent feed that has been previously hydroprocessed, e.g., to reduce the oxygen content of the feed to an appropriately low level. Additionally or alternately, a biocomponent feed can be blended with a mineral feed, so that the blended feed can be tailored to have a relatively low oxygen content, for example bout 500 wppm or less, about 300 wppm or less, about 200 wppm or less, about 100 wppm or less, about 75 wppm or less, about 50 wppm or less, about 35 wppm or less, about 30 wppm or less, about 25 wppm or less, about 20 wppm or less, about 15 wppm or less, about 10 wppm or less, or about 5 wppm or less, optionally in addition to teed targets for sulfur content (as noted herein) and/or nitrogen content (if desired). In embodiments where at least a portion of the feed is of a biocomponent origin, that portion can be at least about 0.2 wt %, for example at least about 0.5 wt %, at least about 1 wt %, at least about 2 wt %, at least about 3 wt %, at least about 5 wt %, at least about 10 wt %, at least about 20 wt %, or at least about 25 wt %. Additionally or alternately, where at least a portion of the feed is of a biocomponent origin, the biocomponent portion can be about 50 wt % or less, for example about 40 wt % or less, about 30 wt % or less, about 25 wt % or less, about 20 wt % or less, about 15 wt % or less, about 10 wt % or less, about 5 wt % or less, about 3 wt % or less, about 2 wt % or less, or about 1 wt % or less.

The contents of components such as sulfur, nitrogen, and oxygen (inter alia) in a feedstream created by blending two or more feedstreams e.g., from similar and/or different sources) can typically be determined using a weighted average based on the blended feeds. For example, a mineral feed and a biocomponent feed can be blended in a ratio of about 80 wt % mineral feed and about 20 wt % biocomponent feed. In such a scenario, if the mineral feed has a sulfur content of about 200 wppm, and the biocomponent feed has a sulfur content of about 10 wppm, the resulting blended feed could be expected to have a sulfur content of about 162 wppm.

The hydrocarbon fuel or fuel precursor feedstream can generally exhibit some measurable sulfur content (i.e., content of sulfur-containing compounds) but advantageously only up to about 100 wppm, for example, from about 2 wppm to about 100 wppm, from about 2 wppm to about 90 wppm, from about 2 wppm to about 80 wppm, from about 2 wppm to about 70 wppm, from about 2 wppm to about 60 wppm, from about 2 wppm to about 50 wppm, from about 2 wppm to about 40 wppm, from about 2 wppm to about 30 wppm, from about 2 wppm to about 20 wppm, from about 2 wppm to about 15 wppm, from about 2 wppm to about 10 wppm, from about 2 wppm to about 8 wppm, from about 2 wppm to about 5 wppm, from about 5 wppm to about 100 wppm, from about 5 wppm to about 90 wppm, from about 5 wppm to about 80 wppm, from about 5 wppm to about 70 wppm, from about 5 wppm to about 60 wppm, from about 5 wppm to about 50 wppm, from about 5 wppm to about 40 wppm, from about 5 wppm to about 30 wppm, from about 5 wppm to about 20 wppm, from about 5 wppm to about 15 wppm, from about 5 wppm to about 10 wppm, from about 5 wppm to about 8 wppm, from about 10 wppm to about 100 wppm, from about 10 wppm to about 90 wppm, from about 10 wppm to about 80 wppm, from about wppm to about 70 wppm, from about 10 wppm to about 60 wppm, from about 10 wppm to about 50 wppm, from about 10 wppm to about 40 wppm, from about wppm to about 30 wppm, from about to 10 wppm to about 20 wppm, from about 10 wppm to about 15 wppm, from about 15 wppm to about 100 wppm, from about 15 wppm to about 90 wppm, from about wppm to about 80 wppm, from about 15 wppm to about 70 wppm, from about 15 wppm to about 60 wppm, from about 15 wppm to about 50 wppm, from about 15 wppm to about 40 wppm, from about 15 wppm to about 30 wppm, from about 30 wppm to about is 100 wppm, from about 30 wppm to about 80 wppm, from about 30 wppm to about 60 wppm, or from about 50 wppm to about 100 wppm.

Although the present invention may not necessarily require a specification on nitrogen content, the hydrocarbon fuel or fuel precursor feedstream may optionally exhibit a nitrogen content (i.e., content of nitrogen-containing compounds) up to about 300 wppm, for example, up to about 200 wppm, up to about 150 wppm, up to about 100 wppm, up to about 80 wppm, up to about 70 wppm, up to about 60 wppm, up to about 50 wppm, up to about 40 wppm, up to about 30 wppm, up to about 25 wppm, up to about 20 wppm, up to about 15 wppm, up to about 10 wppm, up to about 8 wppm, up to about 7 wppm, up to about 6 wppm, up to about 5 wppm, up to about 4 wppm, up to about 3 wppm, up to about 2 wppm, from about 1 wppm to about 100 wppm, from about 1 wppm to about 80 wppm, from about 1 wppm to about 60 wppm, from about 1 wppm to about 50 wppm, from about 1 wppm to about 40 wppm, from about 1 wppm to about 30 wppm, from about 1 wppm to about 20 wppm, from about 1 wppm to about 10 wppm, from about 1 wppm to about 5 wppm, from about 5 wppm to about 100 wppm, from about 5 wppm to about 80 wppm, from about 5 wppm to about 60 wppm, from about 5 wppm to about 50 wppm, from about 5 wppm to about 40 wppm, from about 5 wppm to about 30 wppm, from about 5 wppm to about 20 wppm, from about 5 wppm to about 10 wppm, from about 10 wppm to about 100 wppm, from about 10 wppm to about 80 wppm, from about 10 wppm to about 60 wppm, from about 10 wppm to about 50 wppm, from about 10 wppm to about 40 wppm, from about 10 wppm to about 30 wppm, from about 10 wppm to about 20 wppm, from about 20 wppm to about 100 wppm, from about 20 wppm to about 80 wppm, from about 20 wppm to about 60 wppm, from about 20 wppm to about 50 wppm, from about 20 wppm to about 40 wppm, from about 30 wppm to about 100 wppm, from about 30 wppm to about 80 wppm, from about 30 wppm to about 60 wppm, or from about 50 wppm to about 100 wppm.

In order to attain the sulfur, nitrogen, and/or oxygen contents recited herein, the hydrocarbon fuel or fuel precursor feedstream generally is not a virgin (untreated) feedstream, though virgin feedstreams may be used in the methods according to the invention provided that their components do not significantly overwhelm and/or poison the sulfur sorbent material, thus significantly shortening its usable lifetime, its effectiveness, its capacity, and/or the like. Indeed, typically the hydrocarbon fuel or fuel precursor feedstream can be a product of one or more pre-treatment steps designed, e.g., to remove (for instance common/known) catalyst/sorbent poisons, to lower the sulfur content (and/or the nitrogen content, the oxygen content, and/or any other heteroatom content, as desired) to an appropriately low level but, in the case of sulfur, still above a desired specification, to remove undesirable by-products and/or compounds (e.g., other than heteroatom-containing compounds, such as diolefins, alkynes, aromatics, polynuclear aromatics, paraffins, or the like, or combinations thereof, which specific compounds may depend heavily upon the), to improve some desirable property for the resulting the hydrocarbon fuel product, or the like, or some combination thereof. As a result, prior to being contacted with the sulfur sorbent material, the hydrocarbon fuel or fuel precursor feedstream may first have been subject to one or more of the following steps: hydrotreatment; hydrocracking; hydroconversion; isomerization/dewaxing; aromatic saturation; some other form of hydroprocessing; fluid catalytic cracking; steam cracking; dehumidification/drying; demetallation; deasphalting; coking; or a combination thereof.

One option for pre-treating a feedstream is hydrotreatment, which can include exposing the feedstream to one or more beds of catalyst in one or more hydrotreatment stages. A hydrotreatment process can typically involve exposing a feed to a catalyst in the presence of a hydrogen-containing treat gas. In some embodiments, the hydrotreating catalyst can include, but is not necessarily limited to, a Group VIB metal and/or a Group VIII metal, optionally deposited on a support, Suitable supports can include relatively low acidic metal oxides such as silica, alumina, silica-aluminas, titania, zirconia, or the like, or combinations thereof. The supported Group VIII and/or Group VIB metal(s) can include, but are not limited to, Co, Ni, Fe, Mo, W, Pt, Pd, Rh, Ir, and combinations thereof. Individual hydrogenation metal embodiments can include, but are not limited to, Pt only, Pd only, or Ni only, while mixed hydrogenation metal embodiments can include, but are not limited to, Pt and Pd, Pt and Rh, Ni and W, Ni and Mo, Ni and Mo and W, Co and Mo, Co and Ni and Mo, Co and Ni and W, or another combination. When only one hydrogenation metal is present, the amount of that hydrogenation metal can be at least about 0.1 wt % based on the total weight of the catalyst, for example at least about 0.5 wt % or at least about 0.6 wt %. Additionally or alternately when only one hydrogenation metal is present, the amount of that hydrogenation metal can be about 5.0 wt % or less based on the total weight of the catalyst, for example about 3.5 wt % or less, about 2.5 wt % or less, about 1.5 wt % or less, about 1.0 wt % or less, about 0.9 wt % or less about 0.75 wt % or less, or about 0.6 wt % or less. Further additionally or alternately when more than one hydrogenation metal is present, the collective amount of hydrogenation metals can be at least about 0.1 wt % based on the total weight of the catalyst, for example at least about 0.25 wt %, at least about 0.5 wt %, at least about 0.6 wt %, at least about 0.75 wt %, or at least about 1 wt %. Still further additionally or alternately when more than one hydrogenation metal is present, the collective amount of hydrogenation metals can be about 35 wt % or less based on the total weight of the catalyst, for example about 30 wt % or less, about 2.5 wt % or less, about 20 wt % or less, about 15 wt % or less, about 10 wt % or less, or about 5 wt % or less. In embodiments wherein the supported metal comprises a noble metal, the amount of noble metals) is typically less than about 2 wt %, for example less than about 1 wt %, about 0.9 wt % or less, about 0.75 wt % or less, or about 0.6 wt % or less. The amounts of metal(s) may be measured by methods specified by ASTM for individual metals, including but not limited to atomic absorption spectroscopy (AAS), inductively coupled plasma-atomic emission spectrometry (ICP-AAS), or the like. In some embodiments, multiple beds of catalyst can be used, with each bed of catalyst being the same or different as each other bed of catalyst. Multiple hydrotreatment stages can also be used within a reactor.

The reaction conditions in a hydrotreatment stage can be conditions suitable for reducing the sulfur (and/or other heteroatom) content of the feedstream. For instance, the reaction conditions can include one or more of: an LHSV from about 0.05 hr⁻¹ to about 20 hr⁻¹, for example from about 0.1 hr⁻¹ to about 10 hr, from about 0.3 hr⁻¹ to about 5.0 hr⁻¹, or from about 0.5 hr⁻¹ to about 1.5 hr⁻¹; a total hydrogen pressure from about 250 psig (about 1.7 MPag) to about 5000 psig (about 34 MPag), for example from about 500 psig (about 3.4 MPag) to about 3000 psig (about 21 MPag) or from about 1400 psig (about 9.7 MPag) to about 2000 psig (about 14 MPag); a hydrogen treat gas ratio (based on ˜100% hydrogen; if the treat gas contains one or more diluent gases, the overall treat gas ratio can be proportionally higher) from about 100 scf/bbl (17 Nm³/m³) to about 5000 scf/bbl (840 Nm³/m³); a hydrogen treat gas ratio from about two to about five times the amount of hydrogen to be consumed per barrel of fresh feed in the stage, for example from about four to about five times the amount of hydrogen to be consumed; and a temperature from about 500° F. (about 260° C.) to about 800° F. (about 427° C.), for example from about 600° F. (about 316° C.) to about 775° F. (about 413° C.) or from about 700° F. (about 371° C.) to about 750° F. (about 399° C.).

After a hydrotreatment process, a hydrotreated fled having a significant biocomponent portion can tend to have increased similarity to a hydrotreated purely mineral feed. However, the hydrotreated biocomponent portion (and thus, the mixed feed) can typically have less favorable cold flow properties relative to a comparable hydrotreated purely mineral feed. While the hydrotreated biocomponent feed can have the viscosity characteristics of, e.g., a diesel fuel, the cold flow properties can often restrict the use of a hydrotreated biocomponent feed to, for example, a diesel fuel suitable only for warm weather applications.

The conditions in the hydrotreatment stages, in some embodiments where some level of hydrocracking/conversion is desired, can be effective to convert at least a portion of the feedstock into lower boiling compounds. In an embodiment, the hydrotreatment stages can convert at least about 5 wt % of compounds in the feed boiling above about 355° C. into compounds boiling below about 355° C. for example at least about 10 wt % or at least about 15 wt % of compounds in the feed. Additionally or alternately, the hydrotreatment stages can convert about 30 wt % or less of compounds in the feed boiling above about 355° C. into compounds boiling below about 355° C., for example about 25 wt % or less or about 20 wt % or less.

An additional or alternate option for pre-treating a feedstream can thus include one or more hydrocracking or conversion stages. In such situations, the input feed to the conversion stages can sometimes include a portion of the fractionated bottoms of the effluent from the desulfurization stages. In an embodiment, the hydrocracking or conversion input feed can have an initial boiling point of about 355° C. or greater, for example about 370° C. or greater or about 380° C. or greater. Additionally, or alternately, the hydrocracking or conversion input feed can have a T5 boiling point of about 355° C. or greater, for example about 370° C. or greater or about 380° C. or greater.

In some embodiments, a catalyst in the conversion stages can comprise, consist essentially of or consist of a hydrocracking catalyst. The hydrocracking catalyst can be included as part of a bed and/or stage that contains hydrotreatment catalyst, as mentioned above, or the hydrocracking catalyst can be included in a separate bed and/or stage within, preceding, and/or following, or totally independent of, any hydrotreatment stages % Examples of hydrocracking catalysts can include, but are not limited to, supported catalysts containing nickel, nickel-cobalt-molybdenum, cobalt-molybdenum, tungsten, or nickel-molybdenum components deposited thereon. In another embodiment, the hydrocracking catalyst can include nickel and at least one of tungsten and molybdenum, Other examples of hydrocracking catalysts can include noble metal catalysts, non-limiting examples of which are those based on platinum and/or palladium. Porous support materials, which may be used for both the noble and non-noble metal hydrocracking catalysts can comprise a refractory oxide material including, hut not limited to, alumina, silica, alumina-silica, kieselguhr, diatomaceous earth, magnesia, zirconia, or a combination thereof with alumina, silica, and alumina-silica being preferred and most common), Zeolitic supports including the large pore faujasites such as USY can additionally or alternately be used. In an embodiment, the hydrocracking conditions can be selected based on the hydrotreating conditions. In another embodiment, the hydrotreating conditions can be selected based on effective hydrocracking conditions. Suitable hydrocracking conditions can include one or more of a temperature from about 200° C. to about 450° C., a total pressure from about 5 barg (about 0.5 MPag) to about 300 barg (about 30 MPag), (when hydrogen is present) a hydrogen-containing treat gas ratio from about 100 scf/bbl (about 17 Nm³/m³) to about 5000 scf/bbl (about 840 Nm³/m³), and an LHSV from about 0.05 hr⁻¹ to about 10 hr⁻¹.

The reaction conditions in the conversion stages can be reaction conditions suitable for converting at least a portion of the feed that has a boiling point above about 355° C. to components having a boiling point of about 355° C. or less. Additionally or alternately, the boiling point for measuring the conversion can be based on the initial boiling point (or the T5 boiling point) of the portion of the bottoms fraction that is recycled to the conversion stages. In an embodiment, the reaction conditions can be selected so that the overall conversion of the feedstock from both the hydrocracking (and, if present, additionally the hydrotreatment) stage(s) can be at least about 10%, for example at least about 20%, at least about 30%, at least about 40%, at least about 50%, a least about 60%, or at least about 70%. Additionally or alternately, the overall conversion of the feedstock from both the desulfurization and conversion stages can be about 90% or less, for example about 80% or less, about 70% or less, about 60% or less, about 50% or less, about 40% or less, about 30% or less, or about 20% or less. Suitable conversion conditions can include one or more of a temperature from about 200° C. to about 450° C., a total pressure from about 5 barg (about 0.5 MPag) to about 300 barg (about 30 MPag), (when hydrogen is present) a hydrogen-containing treat gas ratio from about 100 scf/bbl (about 17 Nm³/m³) to about 5000 scf/bbl (about 840 Nm³/m³), and an LHSV from about 0.05 hr⁻¹ to about 10 hr⁻¹.

Another additional or alternate option for pre-treating a feedstream can include one or more isomerization/dewaxing stages. The one or more isomerization/dewaxing stages can, in some embodiments, be included in/with any hydrocracking/conversion stages and/or any hydrotreatment stages. In such situations, an isomerization/dewaxing catalyst can be used in a stage operated under effective hydrocracking conversion conditions and/or under effective hydrotreatment conditions. Using an isomerization/dewaxing catalyst in a hydrocracking/conversion stage, for example, can provide the added benefit of isomerizing the feed during hydrocracking, which can produce additional benefits, particularly regarding the cold flow properties of the hydrocracking/conversion stage effluent. Suitable dewaxing/isomerization catalysts can include, but are not limited to, molecular sieves such as crystalline aluminosilicates (zeolites) or silica-atuminophosphates (SAPOs). In an embodiment, the molecular sieve can comprise or be ZSM-5, ZSM-23, ZSM-35, ZSM-48, zeolite Beta, or a combination thereof, for example ZSM-23 and/or ZSM-48. Additionally or alternately, the molecular sieve can comprise or be a 10-member ring 1-D molecular sieve. Optionally, the to dewaxing/isomerization catalyst can include a binder for the molecular sieve such as those mentioned hereinabove, for instance alumina, titania, silica, silica-alumina, zirconia, or a combination thereof. In an embodiment, the binder can be alumina, titania, or a combination thereof; in another embodiment, the binder can be titania, silica, zirconia, or a combination thereof.

One characteristic of molecular sieves that can impact the activity of the molecular sieve is its ratio of silica to alumina (Si/Al₂). In one embodiment, the molecular sieve can have a silica to alumina ratio of about 200:1 or less, for example about 120:1 or less, about 100:1 or less, about 90:1 or less, or about 75:1 or less. Additionally or alternately, the molecular sieve can have a silica to alumina ratio of at least about 30:1, for example at least about 45:1, at least about 50:1, at least about 55:1, at least about 60:1, at least about 65:1, at least about 70:1, or at least about 75:1.

The dewaxing/isomerization catalyst can also generally include a metal hydrogenation component, such as a Group VIII metal. Suitable Group VIII metals can include Pt, Pd, Ni, Co, or combinations thereof. When present, the Group VIII metal can comprise at least about 0.1 wt % of the catalyst weight, for example at least about 0.3 wt %, at least about 0.5 wt %, at least about 1.0 wt %, at least about 2.0 wt %, at least about 2.5 wt %, at least about 3.0 wt %, at least about 4.0 wt %, or at least about 5.0 wt %. Additionally or alternately, the Group VIII metal can comprise about 15 wt % or less of the catalyst weight, for example about 10 wt % or less, about 5.0 wt % or less, about 4.0 wt % or less, about 3.0 wt % or less, about 2.5 wt % or less, about 2.0 wt % or less, or about 1.5 wt % or less.

In some embodiments, in addition to a Group VIII hydrogenation metal, the dewaxing/isomerization catalyst can also include a Group VIB metal, such as W and/or Mo. When present, typically in combination with a Group VIII metal, the catalyst can include at least about 0.5 wt % of the Group VIB metal, for example at least about 1.0 wt %, at least about 2.0 wt %, at least about 2.5 wt %, at least about 3.0 wt %, at least about 4.0 wt %, or at least about 5.0 wt %. Additionally or alternately, the Group VIII metal can comprise about 20 wt % or less of the catalyst weight, for example about 15 wt % or less, about 10 wt % or less, about 5.0 wt % or less, about 4.0 wt % or less, about 3.0 wt % or less, about 2.5 wt % or less, about 2.0 wt % or less, about 1.5 wt % or less, or about 1.0 wt % or less. In one embodiment, the catalyst can include Pt, Pd, or a combination thereof. In another embodiment, the catalyst can include Ni and W, Ni and Mo, or Ni and a combination of W and Mo.

In embodiments where one or more hydroprocessing pre-treatment steps are performed, it can be beneficial for the feed to those pre-treatment stages to have at least a minimum sulfur content, e.g., sufficient to maintain the sulfided metals of the catalyst(s) in a sulfided (active) state. In such embodiments, for example, the feedstream to be pre-treated can have a sulfur content of at least about 100 wppm, for example at least about 150 wppm or at least about 200 wppm. Additionally or alternately in such embodiments, the feedstock can have a sulfur content of about 500 wppm or less, or about 400 wppm or less, or about 300 wppm or less. In combination with, or regardless of, the sulfur content of the feedstock, additional sulfur can be provided to maintain the metals of the pre-treatment catalyst(s) in a sulfide state, e.g., by introducing gas phase sulfur such as H₂S. One potential source of H₂S gas can be from hydrotreatment of at least the portion of feed originating from a mineral source.

Still another additional or alternate option for pre-treating a feedstream can include one or more aromatic saturation stages containing aromatic saturation (or hydrofinishing) catalysts. Suitable aromatic saturation catalyst can comprise, consist essentially of, or consist of a Group VIII and/or Group VIB metal on a support material, e.g., an amorphous support such as a bound support from the M41S family, for instance bound MCM-41. In some cases, certain hydrotreatment catalysts (as described below) can also be used as aromatic saturation catalysts. The M41S family of catalysts can be described as mesoporous materials having relatively high silica contents, e.g., whose preparation is further described in J. Amer. Chem. Soc., 1992, 114, 10834. Examples of M41S materials can include, but are not limited to MCM-41, MCM-48, MCM-50, and combinations thereof. Mesoporous is understood to refer to catalysts having pore sizes from about 15 Angstroms to about 100 Angstroms. A preferred member of this class is MCM-41, whose preparation is described, e.g., in U.S. Pat. No. 5,098,684. MCM-41 is an inorganic, porous, non-layered phase having a hexagonal arrangement of uniformly-sized pores. The physical structure of MCM-41 is similar to a bundle of straws, in which the opening of the straws (the cell diameter of the pores) ranges from about 15-100 Angstroms. MCM-48 has a cubic symmetry and is described, for example, in U.S. Pat. No. 5,198,203. MCM-50 has a lamellar structure.

MCM-41 can be made with different size pore openings in the mesoporous range. If binders are desired to be used, suitable binders for the M41S family, and specifically for MCM-41, can include alumina, silica, titania, silica-aluminas, or a is combination thereof. Relatively high specific surface areas are possible with such catalysts, such that, in one embodiment, the surface area of the catalyst can be at least about 500 m²/g, for example at least about 600 m²/g. In some embodiments, an even higher surface area catalyst can be selected to further facilitate the aromatic saturation process, for example at least about 750 m²/g, at least about 850 m²/g, or at least about 950 m²/g.

One example of a suitable aromatic saturation catalyst is an alumina-bound mesoporous MCM-41 with a supported hydrogenation metal thereon/therein, e.g., Pt, Pd, another Group VIII metal, a Group VIB metal, or a mixture thereof. Individual hydrogenation metal embodiments can include, but are not limited to, Pt only, Pd only, or Ni only, while mixed hydrogenation metal embodiments can include, but are not limited to, Pt and Pd, Ni and W, Ni and Mo, Ni and Mo and W, Co and Mo, Co and Ni and Mo, or another combination. When present, the amount of Group VIII hydrogenation metal(s) can be at least about 0.1 wt % based on the total weight of the catalyst, for example at least about 0.5 wt % or at least about 0.6 wt %. Additionally or alternately, the amount of Group VIII hydrogenation metal(s) can be about 5.0 wt % or less based on the total weight of the catalyst, for example about 3.5 wt % or less, about 2.5 wt % or less, about 1.5 wt % or less, about 1.0 wt % or less, about 0.9 wt % or less, about 0.75 wt % or less, or about 0.6 wt % or less. Further additionally or alternately, the total amount of hydrogenation metal(s) can be at least about 0.1 wt % based on the total weight of the catalyst, for example at least about 0.25 wt %, at least about 0.5 wt %, at least about 0.6 wt %, at least about 0.75 wt %, or at least about 1 wt %. Still further additionally or alternately, the total amount of hydrogenation metal(s) can be about 35 wt % or less based on the total weight of the catalyst, for example about 30 wt % or less, about 25 wt % or less, about 20 wt % or less, about 15 wt % or less, about 10 wt % or less, or about 5 wt % or less.

An aromatic saturation stage can typically operate at conditions including one or more of the following: a temperature from about 150° C. to about 343° C.; an inlet temperature of about 340° C. or less, for example about 320° C. or less, about 300° C. or less, about 280° C. or less, or about 260° C. or less; an inlet temperature of at least about 230° C., for example at least about 250° C. or at least about 275° C.; a total pressure from about 2.9 MPag (about 400 psig) to about 20.8 MPag (about 3000 psig); a liquid hourly space velocity (LHSV) from about 0.1 hr⁻¹ to about 5 hr⁻¹, for example about 0.5 hr⁻¹ to about 3 hr⁻¹; and a hydrogen treat gas rates can be from about 42 Sm³/m³ (about 250 scf/bbl) to about 1700 Sm³/m³ (about 10,000 scf/bbl). In embodiments where the total hydrogen flow is desired to be maintained at a reduced amount, the hydrogen treat gas rate in the aromatic saturation stage(s) can be about 4500 scf/bbl (about 800 Sm³/m³) or less, for example about 4000 scf/bbl (about 680 Sm³/m³) or less, about 3500 scf/bbl (600 Sm³/m³) or less, or about 3000 scf/bbl (510 Sm³/m³) or less. Additionally or alternately, the hydrogen treat gas rate in the aromatic saturation stage(s) can be at least about 500 scf/bbl (about 85 Sm³/m³), for example at least about 750 scf/bbl (about 130 Sm³/m³) or at least about 1000 scf/bbl (about 170 Sm³/m³).

The one or more aromatic saturation stages can, in some embodiments, immediately follow any isomerization/dewaxing stages. In such situations, the temperature at the inlet of the aromatic saturation stage(s) can advantageously be at least about 20° C. lower than the temperature at the inlet of the isomerization/dewaxing stage(s), for example at least about 25° C. lower, at least about 30° C. lower, at least about 35° C. lower, or at least about 40° C. lower. Additionally or alternately, the temperature at the inlet of the aromatic saturation stage(s) can be at least about 20° C. lower than the temperature at the outlet of the isomerization/dewaxing stage(s), for example at least about 25° C. lower, at least about 30° C. lower, at least about 35° C. lower, at least about 40° C. lower, at least about 45° C. lower, or at least about 50° C. lower.

The sulfur sorbent material can comprise an active copper component, for example, a copper (1) component and/or a copper (II) component. Nevertheless, although the sulfur sorbent material is described herein as comprising an active copper component, the active component can alternatively (or additionally) take the form of another metal that has sulfur sorbent activity, e.g., an active silver component such as a silver (I) component, or the like.

In addition, the sulfur sorbent material can further comprise a porous support. Such support may comprise a zeolite, a mesoporous material, or a combination thereof. Such porous supports may include, but are not necessarily limited to, framework types AFS (such as MAPO-46), ATS (such as a MAPO-36 and/or SSZ-55), BEA (such as zeolite beta), BOG (such as boggsite), CON (such as CIT-1, SSZ-26, and/or SSZ-33), DFO (such as DAF-1), EMT (such as EMC-2), EON (such as ECR-1 and/or TNU-7), ETR (such as ECR-34), EZT (such as EMM-3), FAU (such as zeolite Y, zeolite X, and/or SAPO-37), EMT-FAU intermediates (such as CSZ-1, ECR-30, ZSM-20, and/or ZSM-3), GME (such as gmelinite), LYE (such as zeolite L, perlialite, and/or LZ-212), MAZ (such as mazzite, LZ-202, omega, and/or ZSM-4), MEI (such as ZSM-18 and/or ECR-40), MOR (such as mordenite), MOZ (such as ZSM-10), MSE (such as MCM-68), OFF (such as offretite and/or LZ-217), SAO (such as STA-1), SFO (such as SSZ-51), and/or UFI (such as UZM-5), as well as one or more of MCM-41, SBA-15, and other mesoporous materials, and the like, and combinations and/or structural intermediates thereof. Without being bound by theory, it is believed that exemplary support materials can have relatively high amounts/densities of sites available for exchange with copper to form the active copper component, which exchange sites are also relatively accessible to sulfur-containing species from the feedstream. Such theory, therefore, indicates that support materials having relatively low silica-to-alumina ratios, for example, with either relatively large pore dimensions or with an abundance of surface-accessible exchange sites, can be candidate support materials for use in the methods according to the present invention. In a particular embodiment, the sulfur sorbent material can comprise, consist essentially of, or consist of an active copper (I) component disposed on an FAU framework type zeolite, such as zeolite Y. In another particular embodiment, the sulfur sorbent material useful in the methods according to the invention can be substantially similar to the sorbents disclosed in U.S. Patent Application Publication No. 2009/0118528 A 1, the entirety of which is hereby incorporated herein by reference.

The sulfur removal methods, and/or the sulfur sorbent material used therein, according to the invention can advantageously remove/absorb/adsorb enough of the sulfur-containing compounds present (typically) in hydrocarbon fuels/precursors, e.g., boiling in the naphtha to diesel range(s), to attain the requisite/desired sulfur content and/or percentage sulfur removal. Non-limiting examples of sulfur-containing compounds that are (typically) present in such hydrocarbon fuels/precursors and/or that are removable/absorbable/absorbable by the sulfur sorbent material can include alkyl mercaptans such as DMDS and/or butyl thiols, thiophanes, thiacyclohexanes, thiaindans, carbon disulfide, thiacycloalkanes, thiabicycloalkanes, thiatricycloalkanes, thiatetracycloalkanes, thiophenes, alkylthiophenes, cycloalkylthiophenes, dicycloalkylthiophenes, dithiophenes, alkylthiphenes, benzothiophenes, alkylbenzothiophenes, cycloalkylbenzothiophenes, dicycloalkylbenzothiophenes, dibenzothiophenes, and the like), and the like, and combinations thereof.

The conditions for contacting the hydrocarbon fuel or fuel precursor feedstream with the sulfur sorbent material can be sufficient to reduce the sulfur content of the feedstream by at least about 20 wt %, for example by at least about 25 wt %, by at least about 30 wt %, by at least about 35 wt %, by at least about 40 wt %, by at least about 45 wt %, by at least about 50 wt %, by at least about 55 wt %, by at least about 60 wt %, by at least about 65 wt %, by at least about 70 wt %, by at least about 75 wt %, by at least about 80 wt %, by at least about 85 wt %, by at least about 90 wt %, by at least about 95 wt %, or by at least about 97 wt %; additionally or alternately, conditions for contacting the hydrocarbon fuel or fuel precursor feedstream with the sulfur sorbent material can be sufficient to reduce the sulfur content of the feedstream by about 20 to 98 wt %, for example by about 20 to 95 wt %, by about 20 to 90 wt %, by about 20 to 80 wt %, by about 20 to 65 wt %, by about 20 to 50 wt %, by about 30 to 98 wt %, by about 30 to 95 wt %, by about 30 to 90 wt %, from 30 to 80 wt %, by about 30 to 65 wt %, by about 30 to 50 wt %, by about 40 to 98 wt %, by about 40 to 95 wt %, by about 40 to 90 wt %, by about 40 to 80 wt %, by about 40 to 70 wt %, by about 40 to 60 wt %, by about 40 to 50 wt %, by about 50 to 98 wt %, by about 50 to 95 wt %, by about 50 to 90 wt %, by about 50 to 80 wt %, by about 50 to 70 wt %, by about 50 to 60 wt %, by about 60 to 98 wt %, by about 60 to 95 wt %, by about 60 to 90 wt %, by about 60 to 85 wt %, by about 60 to 80 wt %, by about 60 to 75 wt %, by about 60 to 70 wt %, by about 70 to 98 wt %, by about 70 to 95 wt %, by about 70 to 90 wt %, by about 70 to 85 wt %, by about 70 to 80 wt %, by about 80 to 98 w by about 80 to 95 wt %, by about 80 to 90 wt %, by about 85 to 98 wt %, by about 85 to 95 wt %, or by about 90 to 98 wt %.

The conditions for contacting the hydrocarbon fuel or fuel precursor feedstream with the sulfur sorbent material can advantageously include a temperature of about 392° F. (about 200° C.) or less, for example, about 347° F. (about 175° C.) or less, about 302° F. (about 150° C.) or less, about 257° F. (about 125° C.) or less, about 212° F. (about 100° C.) or less, about 185° F. (about 85° C.) or less, about 167° F. (about 75° C.) or less, about 149° F. (about 65° C.) or less, about 1.22° F. (about 50° C.) or less, or about 86° F. (about 30° C.) or less.

Additionally or alternately, the conditions for contacting the hydrocarbon fuel or fuel precursor feedstream with the sulfur sorbent material can optionally include a pressure at which the hydrocarbon fuel or fuel precursor feedstream remains substantially liquid, or, in terms of measurable values, not higher than about 800 psig (about 5.5 MPag), for example not higher than about 750 psig (about 5.2 MPag), not higher than about 700 psig (about 4.8 MPag), not higher than about 600 psig (about 4.1 MPag), not higher than about 500 psig (about 15 MPag), not higher than about 400 psig (about 2.8 MPag), not higher than about 300 psig (about 2.1 MPag), not higher than about 200 psig (about 1.4 MPag), or not higher than about 100 psig (about 0.7 MPag). In cases where one goal is to operate the process such that the hydrocarbon fuel or fuel precursor feedstream remains substantially liquid, it is noted that, although the hydrocarbon fuel/precursor feedstream may enter in a substantially liquid state, there may additionally be some level of dissolved and/or gas phase by-product (e.g., one or more contaminants from pre-treatment steps such as H₂S, NH₃, or the like, one or more by-products from pre-treatment steps, such as LPG compounds or the like, one or more unreacted components, from pre-treatment steps and/or from the feedstream source, such as hydrogen gas or the like, or any combination thereof) in the feedstream without departing from the “substantially liquid” requirement.

Further additionally or alternately, the conditions for contacting the hydrocarbon fuel or fuel precursor feedstream with the sulfur sorbent material can optionally but preferably include an average contact/residence time that is sufficient to remove the desired amount of sulfur-containing compounds, for example less than about 24 hours, less than about 18 hours, less than about 12 hours, less than about 6 hours, less than about 4 hours, less than about 3 hours, less than about 2 hours, less than about 1.5 hours, less than about 1 hour, less than about 45 minutes, less than about 30 minutes, less than about 20 minutes, less than about 15 minutes, less than about 10 minutes, less than about 5 minutes, less than about 3 minutes, less than about 2 minutes, or less than about 1 minute. Still further additionally or alternately, the conditions for contacting the hydrocarbon fuel or fuel precursor feedstream with the sulfur sorbent material can optionally but preferably include an average contact/residence time of at least about 5 seconds, at least about 10 seconds, at least about 15 seconds, at least about 20 seconds, at least about 30 seconds, at least about 45 seconds, at least about 1 minute, at least about 2 minutes, at least about 3 minutes, at least about 5 minutes, at least about 10 minutes, at least about 15 minutes, at least about 20 minutes, at least about 30 minutes, at least about 45 minutes, at least about 1 hour, at least about 1.5 hours, at least about 2 hours, or at least about 3 hours.

Although many configurations can be employed to reduce sulfur content in hydrocarbon fuel or fuel precursor feedstreams, the methods of the present invention can be particularly suited to one or more of the following specific configurations, optionally with further integration of pre-treatment and/or post-treatment steps, as desired.

In one embodiment, a diesel fuel/precursor that was subject to pre-treatment hydrotreatment at conditions sufficient to yield an off-spec diesel product having a sulfur content greater than 10 wppm, for example greater than 30 wppm, but still about 100 wppm or less, can then be the feedstream sent to contact the sulfur sorbent material under conditions sufficient to form an on-spec diesel fuel by reducing the sulfur content to 30 wppm or less, for example, in the case of on-spec ultra-low-sulfur diesel, to 10 wppm or less (and optionally, if necessary or desired, by mixing the lower sulfur diesel boiling range product with another fuel/precursor) to form a product meeting the sulfur specification for diesel fuel. This embodiment can be particularly important for diesel boiling range feedstreams that are slightly off—spec on sulfur content. This embodiment can additionally or alternately be particularly important for situations in which the pre-treatment hydrotreatment unit is constrained due to the hydrotreatment catalyst having a relatively low activity, e.g., towards the end of its cycle length—normally, the severity of the hydrotreatment conditions in the pre-treatment step can be ramped up to compensate for low catalytic activity, but this embodiment allows for lower severity hydrotreatment pre-treatment steps, by virtue of a sulfur sorbent post-treatment to pick up the sulfur removal slack. This embodiment can further additionally or alternately be particularly important for situations in which the pre-treatment hydrotreatment unit is constrained due to a desired (or necessary) increase in the unit throughput (feed rate)—again, normally the severity of the hydrotreatment conditions in the pretreatment step can be ramped up to compensate for increased throughput, but this embodiment allows for lower severity hydrotreatment pre-treatment steps, by virtue of a sulfur sorbent post-treatment to pick up the sulfur removal slack. This embodiment can still further additionally or alternately be particularly important for situations in which recycle of off-spec (at least based on sulfur content) diesel fuel through the pre-treatment hydrotreatment process is undesirable or impermissible for some reason (e.g., throughput constraints) yet again, normally the severity of the hydrotreatment conditions in the pre-treatment step can be ramped up to avoid off-spec sulfur diesel fuel, but this embodiment allows for lower severity hydrotreatment pre-treatment steps, by virtue of a sulfur sorbent post-treatment to pick up the sulfur removal slack. This embodiment of a sulfur sorbent step after a hydrotreatment pre-treatment may further be more desirable than increased throughput and/or recycle to the hydrotreatment step because of reduced need for/use of hydrogen gas and/or because of reduced heating duty in the sulfur sorbent step, each/both of which can represent a significant cost savings over time.

In another embodiment, a naphtha fuel/precursor having (and/or optionally subject pre-treatment hydrotreatment at conditions sufficient to yield) an off-spec naphtha product having a sulfur content greater than 10 wppm, for example greater than 15 wppm, but still about 100 wppm or less, can then be the feedstream sent to contact the sulfur sorbent material under conditions sufficient to form an on-spec motor gasoline fuel by reducing the sulfur content to 15 wppm or less, for example, in the case of on-spec ultra-low-sulfur motor gasoline, to 10 wppm or less (and optionally, if necessary or desired, by mixing the lower sulfur naphtha boiling range product with another fuel/precursor) to form a product meeting the sulfur specification for motor gasoline fuel. This embodiment can be particularly important for naphtha boiling range feedstreams that are slightly off-spec on sulfur content. This embodiment can additionally or alternately be particularly important for situations in which the pre-treatment hydrotreatment unit is constrained due to the hydrotreatment catalyst having a relatively low activity, e.g., towards the end of its cycle length—normally, the severity of the hydrotreatment conditions in the pre-treatment step can be ramped up to compensate for low catalytic activity, but this embodiment allows for lower severity hydrotreatment pre-treatment steps, by virtue of a sulfur sorbent post-treatment to pick up the sulfur removal slack. This embodiment can further additionally or alternately be particularly important for situations in which the pre-treatment hydrotreatment unit is constrained due to a desired (or necessary) increase in the unit throughput (feed rate) again, normally the severity of the hydrotreatment conditions in the pre-treatment step can be ramped up to compensate for increased throughput, but this embodiment allows for lower severity hydrotreatment pre-treatment steps, by virtue of a sulfur sorbent post-treatment to pick up the sulfur removal slack. This embodiment can still further additionally or alternately be particularly important for situations in which recycle of off-spec (at least based on sulfur content) motor gasoline fuel through the pre-treatment hydrotreatment process is undesirable or impermissible for some reason (e.g., throughput constraints)—yet again, normally the severity of the hydrotreatment conditions in the pre-treatment step can be ramped up to avoid off-spec sulfur motor gasoline fuel, but this embodiment allows for lower severity hydrotreatment pre-treatment steps, by virtue of a sulfur sorbent post-treatment to pick up the sulfur removal slack. This embodiment of a sulfur sorbent step after a hydrotreatment pre-treatment may further be more desirable than increased throughput and/or recycle to the hydrotreatment step because of reduced need for/use of hydrogen gas and/or because of reduced heating duty in the sulfur sorbent step, each/both of which can represent a significant cost savings over time. Indeed, particularly for fuels/precursors in which higher content of unsaturated components (such as olefins) can be desired (e.g., for naphtha/gasoline, kero, and/or jet precursors/fuels), increased severity and/or increased exposure to hydrotreatment environments can unintentionally result in lower unsaturates content, whereas the sulfur sorbent treatment according to the invention can allow removal of sulfur with incrementally better retention of unsaturates content, even with relatively selective hydrotreatment catalysts/conditions.

In another embodiment, a naphtha fuel/precursor that optionally was subject to pre-treatment hydrotreatment and that is desired to be post-treated in the presence of a catalyst having particular sulfur sensitivity (e.g., where sulfur-containing compounds can immediately or over time poison a post-treatment catalyst) can be the feedstream first sent to contact the sulfur sorbent material under conditions sufficient to reduce the sulfur content to as low as desired (which can depend upon the effect of certain sulfur-containing compounds of the particular catalyst, but generally which can result in a sulfur content of about 10 wppm or less, for example about 8 wppm or less, about 7 wppm or less, about 6 wppm or less, about 5 wppm or less, about 4 wppm or less, about 3 wppm or less, about 2 wppm or less, about 1 wppm or less, about 500 wppb or less, about 300 wppb or less, about 200 wppb or less, or about 100 wppb or less), and then post-treated in a process step using the aforementioned catalyst having particular sulfur sensitivity. Examples of post-treatment processes using catalysts having particular sulfur sensitivity can include, but are in no way limited to, catalytic reforming, C₅/C₆ isomerization, naphtha H₂ plant feed, or the like, or a combination thereof. This embodiment can be particularly important for such post-treatment catalyst sulfur sensitivity situations, e.g., because normally the options for reducing sulfur to such low levels can include compensating by one or more of: increasing the severity of the optional pre-treatment hydrotreatment conditions; reducing the throughput of the optional pre-treatment hydrotreatment conditions; (re)cycling feed having too high a sulfur content to an (the) optional pre-treatment hydrotreatment step (in the recycle case, while not increasing total throughput, thus reducing first pass throughput); using a higher temperature process to attain such low sulfur levels; using a process requiring hydrogen to attain such low sulfur levels, and the like. This embodiment of a sulfur sorbent step before contacting a sulfur-sensitive catalyst may be more desirable than adding a pre-treatment hydrotreatment step, adjusting recycle/conditions/throughput on an existing pre-treatment hydrotreatment step, because of reduced need for/use of hydrogen gas, because of increased flexibility, and/or because of reduced heating duty through use of the sulfur sorbent step, each/some/all of which can represent a significant cost savings over time. Indeed, particularly for fuels/precursors in which higher content of unsaturated components (such as olefins) can be desired (e.g., for naphtha/gasoline, kero, and/or jet precursors/fuels), increased severity and/or increased exposure to hydrotreatment environments can unintentionally result in lower unsaturates content, whereas the sulfur sorbent treatment according to the invention can allow removal of sulfur with incrementally better retention of unsaturates content, even with relatively selective hydrotreatment catalysts/conditions.

In situations where post-treatment catalytic reforming is performed, suitable reforming catalysts can include monofunctional and/or bifunctional catalysts, Monofunctional catalysts can typically include a hydrogenation-dehydrogenation function. Bifunctional catalysts can typically include both a hydrogenation-dehydrogenation function and an acid function. In an embodiment, the reforming catalyst can include at least one metal from Group VIII of the Periodic Table of Elements CAS version). Suitable Group VIII metals can include, but are not limited to, nickel, the noble metals (specifically platinum, palladium, iridium, ruthenium, rhodium, osmium, and combinations thereof), and combinations thereof. Preferred metals, in some embodiments, include the noble metals, particularly platinum. It can additionally or alternately be preferred in some embodiments that the catalyst composition have a relatively high specific surface area, e.g., from about 100 m²/g to about 400 m²/g. In a preferred embodiment, the Group VIII metal can be present on the catalyst in an amount from about 0.01 wt % to about 5 wt %, for example from about 0.1 wt % to about 2 wt %, calculated on an elemental basis, based on the total weight of the final catalytic composition.

In certain embodiments of the invention including post-treatment reforming, the reforming catalyst used can also contain at least one promoter metal from Group IIA (such as gallium), Group IVA (such as tin), Group IIB (such as copper), Group VIB (such as chromium), and Group VIIB (such as rhenium). Preferably, in such embodiments, the reforming catalyst can include one or more of rhenium and tin. When present, the promoter metal can be present in the form of an oxide, sulfide, or elemental stage in an amount from about 0.01 wt % to about 5 wt % for example from about 0.1 wt % to about 3 wt % or from about 0.2 wt % to about 3 wt %, calculated on an elemental basis, based on the total weight of the final catalyst composition.

In certain embodiments, the catalyst can comprise at least one halide component in an amount effective to provide acid functionality. Examples of such a halide component can include fluoride, chloride, iodide, bromide, or a combination thereof. Generally, the amount of halide, when present, can be such that the final catalyst composition contains from about 0.1 wt % to about 3.5 wt %, for example from about 0.5 wt % to about 1.5 wt % of halogen calculated on an elemental basis.

In certain embodiments of the invention including post-treatment reforming, the reforming catalyst used can include a support material, e.g., a refractory support, preferably such as a relatively high surface area material that is relatively uniform in composition without limited composition gradients. Examples of suitable supports can include, but are not limited to, one or more of: (1) refractory inorganic oxides such as alumina, silica, titania, magnesia, zirconia, chromia, thoria, boria, or mixtures thereof; (2) synthetically prepared or naturally occurring clays and silicates; (3) crystalline zeolitic aluminosilicates, either naturally occurring or synthetically prepared, such as FAU, MEL, MFI, MOR, MTW (IUPAC Commission on Zeolite Nomenclature), in hydrogen form or in a form which has been exchanged with metal cations; (4) non-zeolitic molecular sieves; and (5) spinets such as MgAl₂O₄, FeAl₂O₄, ZnAl₂O₄, CaAl₂O₄. In some embodiments, the support material can comprise or can be a crystalline silicate, also referred to as a silicate or zeolite. In such embodiments, the support can preferably be an intermediate or large pore zeolite. In general, such zeolites can have an average pore diameter of more than 5 Å, for example from about 5 Å to about 15 Å. Examples of intermediate pore support materials suitable for use in this invention can include, but are not limited to, ZSM and CZH zeolites, ZSM-5, ZSM-1.1, ZSM-12, ZSM-22, ZSM-23, ZSM-35, ZSM-38, ZSM-48, CZH-5, and the like, and combinations thereof. Examples of large pore support materials suitable for use in this invention can include, but are not limited to, zeolite X, zeolite Y, zeolite L, faujasite, mordenite, and the like, and combinations thereof.

In some embodiments, the reforming catalyst can include a structural support having an average particle diameter of not greater than about 500 microns, preferably not greater than about 300 microns, for example not greater than about 100 microns. Such a catalyst can be particularly advantageous in relatively high space velocity reaction processes.

Additionally or alternately, the present invention can be described according to one or more of the following embodiments.

### Embodiment 1

A method for removing sulfur from a hydrocarbon fuel or fuel precursor feedstream comprising: contacting a hydrocarbon fuel or fuel precursor feedstream having a sulfur content from about 2 wppm to about 100 wppm with a sulfur sorbent material comprising an active copper component disposed on a porous support under conditions sufficient to reduce the sulfur content by at least about 20 wt %, thus forming a hydrocarbon fuel product, wherein the conditions include at least a temperature of about 392° F. (about 200° C.) or less, optionally a pressure at which the hydrocarbon fuel or fuel precursor feedstream remains substantially liquid, and optionally an average contact/residence time of less than about 4 hours, and wherein the porous support is comprised of a zeolite, a mesoporous material, or a combination thereof.

### Embodiment 2

The method of embodiment 1, wherein the hydrocarbon fuel or fuel precursor feedstream comprises a naphtha stream, a gasoline precursor stream, a gasoline fuel stream, a diesel precursor stream, a hydrotreated diesel stream, a diesel fuel stream, a jet fuel precursor stream, a jet fuel stream, a kero precursor stream, a kero fuel stream, or a combination thereof; and wherein the hydrocarbon fuel product comprises a gasoline fuel, a jet fuel, a kerosene fuel, a diesel fuel, or a combination thereof.

### Embodiment 3

The method of any one of the previous embodiments, wherein the hydrocarbon fuel or fuel precursor feedstream exhibits one or more of the following: an MP of at least about 90° F. (about 32° C.); IBP of about 450° F. (about 232° C.) or less; a T5 boiling point of at least about 100° F. (about 38° C.); a T5 boiling point of about 450° F. (about 232° C.) or less; a T95 boiling point of at least about 350° F. (about 177° C.); a T95 boiling point of about 725° F. (about 385° C.) or less; an FBP of at least about 350° F. (about 177° C.); and an FBP of about 750° F. (about 399° C.) or less.

### Embodiment 4

The method of any one of embodiments 1-3, wherein the hydrocarbon fuel or fuel precursor feedstream comprises a naphtha stream, a gasoline precursor stream, a gasoline fuel stream, or a combination thereof, and also exhibits one or more of the following: an IBP of at least about 90° F. (about 32° C.); IBP of about 150° F. (about 66° C.) or less; a T5 boiling point of at least about 100° F. (about 38° C.); a T5 boiling point of about 200° F. (about 93° C.) or less; a T95 boiling point of at least about 325° F. (about 163° C.); a T95 boiling point of about 425° F. (about 218° C.) or less; an FBP of at least about 350° F. (about 177° C.); and an FBP of about 425° F. (about 218° C.) or less.

### Embodiment 5

The method of any one of embodiments 1-3, wherein the hydrocarbon fuel or fuel precursor feedstream comprises a jet fuel precursor stream, a jet fuel stream, a kero precursor stream, a kero fuel stream, or a combination thereof, and also exhibits one or more of the following: an IBP of at least about 150° F. (about 66° C.); an IBP of about 375° F. (about 191° C.) or less; a T5 boiling point of at least about 200° F. (about 93° C.); a T5 boiling point of about 400° F. (about 204° C.) or less; a T95 boiling point of at least about 500° F. (about 260° C.); a T95 boiling point of about 575° F.′ (about 302° C.) or less; an FRP of at least about 500° F. (about 260° C.); and an FBP of about 625° F. (about 329° C.) or less.

### Embodiment 6

The method of any one of embodiments 1-3, wherein the hydrocarbon fuel or fuel precursor feedstream comprises a diesel precursor stream, a hydrotreated diesel stream, a diesel fuel stream, or a combination thereof, and also exhibits one or more of the following: an IBP of at least about 350° F. (about 177° C.); an IBP of about 450° F. (about 232° C.) or less; a T5 boiling point of at least about 375° F. (about 191° C.); a T5 boiling point of about 450° F. (about 232° C.) or less; a T95 boiling point of at least about 650° F. (about 343° C.); a T95 boiling point of about 725° F. (about 385° C.) or less; an FBP of at least about 675° F. (about 357° C.); and an FBP of about 750° F. (about 399° C.) or less.

### Embodiment 7

The method of any one of the previous embodiments, wherein the porous support comprises a zeolite having framework type AFS, ATS, BEA, BOG, CON, DFO, EMT, EON, ETR, EZT, FAU, a structural EMT-FAU intermediate, GME, LTL, MAZ, MEI, MOR, MOZ, MSE, OFF, SAO, SFO, and/or UFI, or a combination or structural intermediate thereof.

### Embodiment 8

The method of any one of the previous embodiments, wherein the porous support comprises a MAPO-46, a MAPO-36, an SSZ-55, a zeolite beta, a boggsite, a CIT-1, a SSZ-26, a SSZ-33, a DAF-1, an EMC-2, an ECR-1, a TNU-7, an ECR-34, an EMM-3, a zeolite Y, a zeolite X, a SAPO-37, a CSZ-1, an ECR-30, a ZSM-20, a ZSM-3, a gmelinite, a zeolite L, a perlialite, an LZ-212, a mazzite, an LZ-202, an omega, a ZSM-4, a ZSM-18, an ECR-40, a mordenite, a ZSM-10, a MCM-68, an offretite, an LZ-217, an STA-1, an SSZ-51, a UZM-5, an MCM-41, an SBA-15, or a combination or structural intermediate thereof.

### Embodiment 9

The method of any one of the previous embodiments, wherein the active copper component comprises an active copper (1) component exchanged onto the porous support.

### Embodiment 10

The method of any one of embodiments 1-4 and 7-9, wherein one or more of the following is satisfied: the hydrocarbon fuel or fuel precursor feedstream exhibits a sulfur content from about 10 wppm to about 50 wppm; the sulfur sorbent material comprises an active copper (1) component disposed on zeolite Y; the contacting conditions are sufficient to reduce the sulfur content of the feedstream by least about 20 to 80 wt %; and the contacting conditions include a temperature of about 302° F. (about 150° C.) or less.

### Embodiment 11

The method of any one of embodiments 1-3 and 6-9, wherein one or more of the following is satisfied: the hydrocarbon fuel or fuel precursor feedstream exhibits a sulfur content from about 15 wppm to about 100 wppm; the sulfur sorbent material comprises an active copper (1) component disposed on zeolite Y; the contacting conditions are sufficient to reduce the sulfur content of the feedstream by least about 30 to 90 wt %; and the contacting conditions include a temperature of about 149° F. (about 65° C.) or less.

### Embodiment 12

The method of any one of the previous embodiments, wherein the hydrocarbon fuel or fuel precursor feedstream comprises at least 5 wt % of a biocomponent feed.

### Embodiment 13

The method of any one of the previous embodiments, wherein the hydrocarbon fuel or fuel precursor feedstream remains as a liquid during the contacting with the sulfur sorbent material.

### Embodiment 14

The method of any one of the previous embodiments, is wherein at least a portion of the hydrocarbon fuel product is contacted with a reforming catalyst comprising at least one metal from Group VIII of the Periodic Table of Elements.

### Embodiment 15

The method of embodiment 14, wherein the Group VIII metal is selected from platinum and palladium.

### Embodiment 16

The method of embodiment 15, wherein the reforming catalyst further comprises at least one promoter metal from Group IIA, Group IVA, Group IB, Group VIB, and Group VIIB of the Periodic Table of Elements.

### Embodiment 17

The method of any one of the previous embodiments, wherein the hydrocarbon fuel or fuel precursor feedstream is a product of a hydrocracking process; wherein the hydrocracking process comprises a hydrocracking catalyst which is a supported catalyst containing nickel, nickel-cobalt-molybdenum, cobalt-molybdenum, nickel-tungsten, or nickel-molybdenum components deposited thereon; and the hydrocracking process is operated at hydrocracking conditions which include a temperature from about 200° C. to about 450° C., a total pressure from about 5 barg (about 0.5 MPag) to about 300 barg (about 30 MPag), a hydrogen-containing treat gas ratio from about 100 scf/bbl (about 17 Nm³/m³) to about 5000 scf/bbl (about 840 Nm³/m³), and an LHSV from about 0.05 hr⁻¹ to about 10 hr⁻¹.

### EXAMPLES

### Example 1

### Preparation and Regeneration of Cu(I) on Zeolite Y Sulfur Sorbent Material

Example 1 details preparation methods for forming Cu(I)—Y sulfur sorbent materials according to the invention. The preparation methods for Catalysts A and B, made using vapor-phase ion exchange (VPIE) and liquid-phase ion exchange (LPIE) techniques, respectively, were completed experimentally, whereas the preparation methods for Catalysts C-1 (based on liquid-phase ion exchange techniques) are prophetic. See Table 1 below.

TABLE 1

| Catalyst | Technique | Support | Exchange ion | Reduction Conds. | Regen. Conditions |
|---|---|---|---|---|---|
| A | VPIE | Na—Y | Cu(NO₃)₂ | Inert gas (He) @ | Air @ ~350° C. for ~6 hrs., |
| | | | | ~450° C. for ~18 hrs. | then re-reduce |
| B | LPIE | Na—Y | Cu(NO₃)₂ | Inert gas (He) @ | Air @ ~350° C. for ~6 hrs., |
| | | | | ~450° C. for ~18 hrs. | then re-reduce |
| C | LPIE | H—Y | Cu(NO₃)₂ | Inert gas (He) @ | Air @ ~350° C. for ~6 hrs., |
| | | | | ~450° C. for ~18 hrs. | then re-reduce |
| D | LPIE | NH₄—Y | Cu(NO₃)₂ | Inert gas (He) @ | Air @ ~350° C. for ~6 hrs., |
| | | | | ~450° C. for ~18 hrs. | then re-reduce |
| E | LPIE | Na—Y | CuSO₄ | Inert gas (He) @ | Air @ ~350° C. for ~6 hrs., |
| | | | | ~450° C. for ~18 hrs. | then re-reduce |
| F | LPIE | H—Y | CuSO₄ | Inert gas (He) @ | Air @ ~350° C. for ~6 hrs., |
| | | | | ~450° C. for ~18 hrs. | then re-reduce |
| G | LPIE | NH₄—Y | CuSO₄ | Inert gas (He) @ | Air @ ~350° C. for ~6 hrs., |
| | | | | ~450° C. for ~18 hrs. | then re-reduce |
| H | LPIE | Na—Y | Cu(OAc)₂ | Inert gas (He) @ | Air @ ~350° C. for ~6 hrs., |
| | | | | ~450° C. for ~18 hrs. | then re-reduce |
| I | LPIE | H—Y | Cu(OAc)₂ | Inert gas (He) @ | Air @ ~350° C. for ~6 hrs., |
| | | | | ~450° C. for ~18 hrs. | then re-reduce |
| J | LPIE | NH₄—Y | Cu(OAc)₂ | Inert gas (He) @ | Air @ ~350° C. for ~6 hrs., |
| | | | | ~450° C. for ~18 hrs. | then re-reduce |

Vapor-phase ion and liquid-phase ion exchange conditions for the catalyst samples in this Example were (or can be) similar to those disclosed in paragraphs [0041]-[0044] of U.S. Patent Application Publication No. 2009/0118528 A1. It is noteworthy that Catalyst A, made by vapor-phase ion exchange, exhibited almost about twice the copper loading as Catalyst B, made by liquid-phase ion exchange.

### Example 2

### Cu(I)—Y Effectiveness in Sulfur Removal from Naphtha Boiling Range Feed

In Example 2, Catalyst A was used as a sulfur sorbent material in a method according to the invention, in order to reduce the sulfur content of a light naphtha feedstream that had been pre-treatment hydrocracked and spiked with thiophene. The spiked light naphtha feed had an API gravity of about 58.6, a sulfur content of about 9.8 wppm, a nitrogen content of less than 0.2 wppm, a T50 boiling point of about 199° F. (about 93° C.), and a T95 boiling point of about 250° F. (about 121° C.).

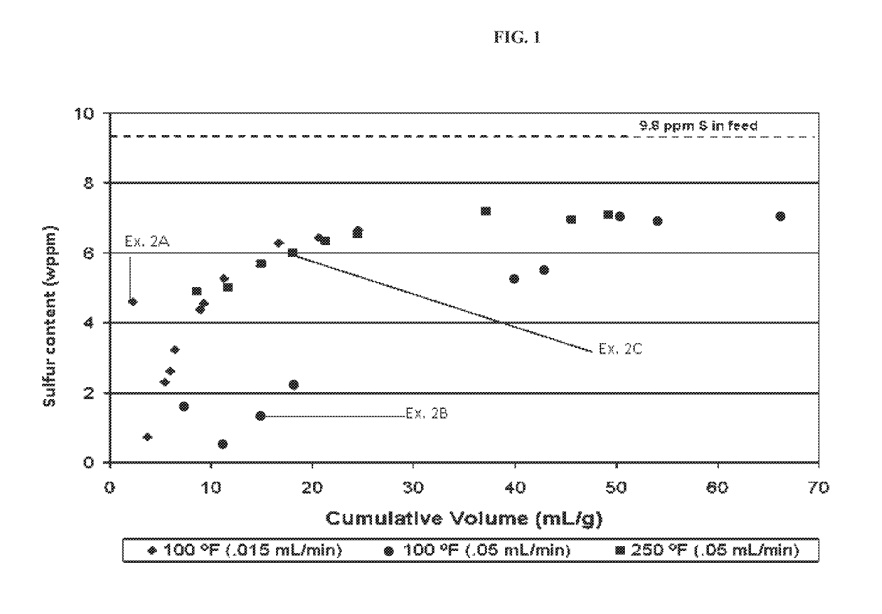

About 1.5-3 grams of Catalyst A sorbent material was contacted with the light naphtha feed at one or two temperatures (either ˜100° F. or ˜250° F.) and at one of is two teed rates (˜0.015 mL/min or ˜0.05 mL/min). Product sampling to determine sulfur content was done at approximately 2-hour intervals, and the data was plotted as wppm sulfur content versus cumulative sulfur volume (mL), normalized per gram of sorbent material. Results are shown in FIG. 1—Example 2A (diamonds) represents a temperature of about 100° F. (about 38° C.) and a feed rate of about 0.015 mL/min; Example 2B (circles) represents a temperature of about 100° F. (about 38° C.) and a feed rate of about 0.05 mL/min; and Example 2C (squares) represents a temperature of about 250° F. (about 121° C.) and a feed rate of about 0.05 mL min.

As can be seen from FIG. 1, it appears that the sorbent material was more effective at trapping sulfur in the light naphtha feed at lower temperatures, which corresponds to substantially liquid phase conditions, and surprisingly at the higher feed rate at those lower temperatures. The sorbent capacity seemed to level out over time to yield a sulfur content of about 7-7.5 wppm, which represents about 25-30% sulfur removal capability at longer time scales.

### Example 3

### Cu(I)—Y Effectiveness in Sulfur Removal from Diesel Boiling Range Feed

In Example 3, Catalyst A was used as a sulfur sorbent material in a method according to the invention, in order to reduce the sulfur content of a diesel feedstream that had been pre-treatment hydrocracked and spiked with 4,6-dibenzothiophene (DBT). The spiked diesel feed had an API gravity of about 40.4, a sulfur content of about 40.8 wppm, a nitrogen content of less than 0.2 wppm, a T50 boiling point of about 499° F. (about 259° C.), and a T95 boiling point of about 689° F. (about 365° C.).

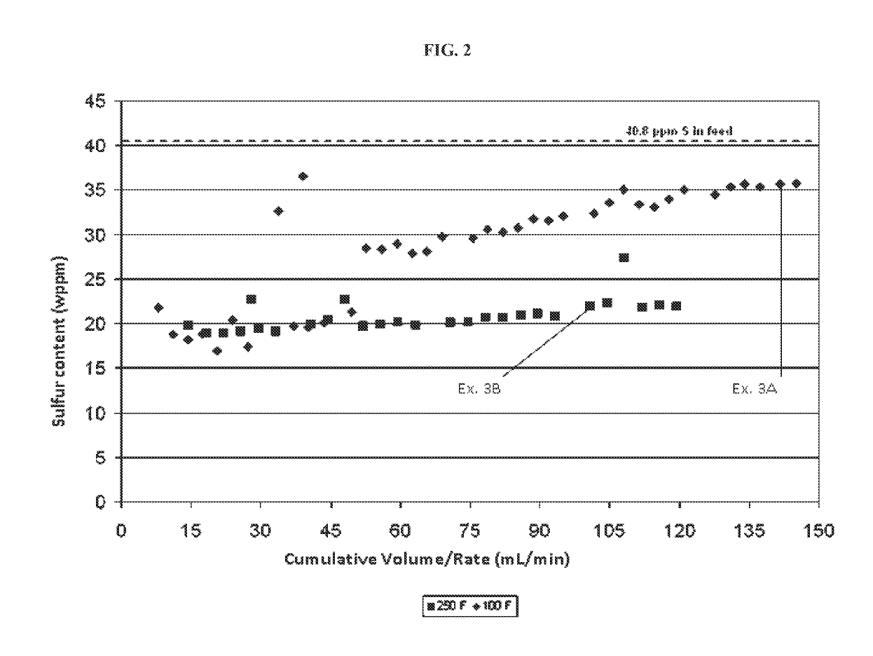

About 3 grams of Catalyst A sorbent material was contacted with the diesel feed at one or two temperatures (either ˜100° F. or ˜250° F.) and at a feed rate of about 0.05 mL/min. Product sampling to determine sulfur content was done at regular intervals, and the data was plotted as wppm sulfur content versus cumulative sulfur volume/rate (mL/min). Results are shown in FIG. 2—Example 3A (diamonds) represents a temperature of about 100° F. (about 38° C.); and Example 3B (squares) represents a temperature of about 250° F. (about 121° C.).

As can be seen from FIG. 2, it appears that the sorbent material at either temperature initially removed about 50% (about 45-60%) of the sulfur content in the diesel feed, but the higher temperature condition resulted in a longer maintenance of this sulfur removal effectiveness than the lower temperature condition.

While the present invention has been described and illustrated by reference to particular embodiments, those of ordinary skill in the art will appreciate that the invention lends itself to variations not necessarily illustrated herein. For this reason, then, reference should be made solely to the appended claims for purposes of determining the true scope of the present invention.

What is claimed is:

1. A method for removing sulfur from a hydrocarbon fuel or fuel precursor feedstream comprising:
contacting a hydrocarbon fuel or fuel precursor feedstream having a sulfur content from about 2 wppm to about 100 wppm with a sulfur sorbent material comprising an active copper component disposed on a porous support under conditions sufficient to reduce the sulfur content by at least about 20 wt %, thus forming a hydrocarbon fuel product,
wherein the conditions include at least a temperature of about 392° F. (about 200° C.) or less, optionally a pressure at which the hydrocarbon fuel or fuel precursor feedstream remains substantially liquid, and optionally an average contact/residence time of less than about 4 hours; and wherein the porous support is comprised of a zeolite, a mesoporous material, or a combination thereof.

2. The method of claim 1, wherein the hydrocarbon fuel or fuel precursor feedstream comprises a naphtha stream, a gasoline precursor stream, a gasoline fuel stream, a diesel precursor stream, a hydrotreated diesel stream, a diesel fuel stream, a jet fuel precursor stream, a jet fuel stream, a kero precursor stream, a kero fuel stream, or a combination thereof; and wherein the hydrocarbon fuel product comprises a gasoline fuel, a jet fuel, a kerosene fuel, a diesel fuel, or a combination thereof.

3. The method of claim 1, wherein the hydrocarbon fuel or fuel precursor feedstream exhibits one or more of the following: an IBP of at least about 90° F. (about 32° C.); IBP of about 450° F. (about 232° C.) or less; a T5 boiling point of at least about 100° F. (about 38° C.); a T5 boiling point of about 450° F. (about 232° C.) or less; a T95 boiling point of at least about 350° F. (about 177° C.); a T95 boiling point of about 725° F. (about 385° C.) or less; an FBP of at least about 350° F. (about 177° C.); and an FBP of about 750° F. (about 399° C.) or less.

4. The method of claim 1, wherein the hydrocarbon fuel or fuel precursor feedstream comprises a naphtha stream, a gasoline precursor stream, a gasoline fuel stream, or a combination thereof, and also exhibits one or more of the following: an IBP of at least about 90° F. (about 32° C.); an IBP of about 150° F. (about 66° C.) or less; a T5 boiling point of at least about 100° F. (about 38° C.); a T5 boiling point of about 200° F. (about 93° C.) or less; a T95 boiling point of at least about 325° F. (about 163° C.); a T95 boiling point of about 425° F. (about 218° C.) or less; an FBP of at least about 350° F. (about 177° C.); and an FBP of about 425° F. (about 218° C.) or less.

5. The method of claim 1, wherein the hydrocarbon fuel or fuel precursor feedstream comprises a jet fuel precursor stream, a jet fuel stream, a kero precursor stream, a kero fuel stream, or a combination thereof, and also exhibits one or more of the following: an IBP of at least about 150° F. (about 66° C.); an IBP of about 375° F. (about 191° C.) or less; a T5 boiling point of at least about 200° F. (about 93° C.); a T5 boiling point of about 400° F. (about 204° C.) or less; a T95 boiling point of at least about 500° F. (about 260° C.); a T95 boiling point of about 575° F. (about 302° C.) or less; an FBP of at least about 500° F. (about 260° C.); and an FBP of about 625° F. (about 329° C.) or less.

6. The method of claim 1, wherein the hydrocarbon fuel or fuel precursor feedstream comprises a diesel precursor stream, a hydrotreated diesel stream, a diesel fuel stream, or a combination thereof, and also exhibits one or more of the following: an IBP of at least about 350° F. (about 177° C.); an IBP of about 450° F. (about 232° C.) or less; a T5 boiling point of at least about 375° F. (about 191° C.); a T5 boiling point of about 450° F. (about 232° C.) or less; a T95 boiling point of at least about 650° F. (about 343° C.); a T95 boiling point of about 725° F. (about 385° C.) or less; an FBP of at least about 675° F. (about 357° C.); and an FBP of about 750° F. (about 399° C.) or less.

7. The method of claim 1, wherein the porous support comprises a zeolite having framework type AFS, ATS, BEA, BOG, CON, DFO, EMT, EON, ETR, EZT, FAU, a structural EMT-FAIT intermediate, GME, LTL, MAZ, MEI, MOR, MOZ, MSE, OFF, SAO, SFO, and/or UFI, or a combination or structural intermediate thereof.

8. The method of claim 1, wherein the porous support comprises a MAPO-46, a MAPO-36, an SSZ-55, a zeolite beta, a boggsite, a CIT-1, a SSZ-26, a SSZ-33, a DAF-1, an EMC-2, an ECR-1, a TNU-7, an ECR-34, an EMM-3, a zeolite Y, a zeolite X, a SAPO-37, a CSZ-1, an ECR-30, a ZSM-20, a ZSM-3, a gmelinite, a zeolite L, a perlialite, an LZ-212, a mazzite, an LZ-202, an omega, a ZSM-4, a ZSM-18, an ECR-40, a mordenite, a ZSM-10, a MCM-68, an offretite, an LZ-217, an STA-1, an SSZ-51, a UZM-5, an MCM-41, an SBA-15, or a combination or structural intermediate thereof.

9. The method of claim 1, wherein the active copper component comprises an active copper (I) component exchanged onto the porous support.

10. The method of claim 4, wherein one or more of the following is satisfied: the hydrocarbon fuel or fuel precursor feedstream exhibits a sulfur content from about 10 wppm to about 50 wppm; the sulfur sorbent material comprises an active copper (I) component disposed on zeolite Y; the contacting conditions are sufficient to reduce the sulfur content of the feedstream by at least about 20 to 80 wt %; and the contacting conditions include a temperature of about 302° F. (about 150° C.) or less.

11. The method of claim 6, wherein one or more of the following is satisfied: the hydrocarbon fuel or fuel precursor feedstream exhibits a sulfur content from about 15 wppm to about 100 wppm; the sulfur sorbent material comprises an active copper (I) component disposed on zeolite Y; the contacting conditions are sufficient to reduce the sulfur content of the feedstream by at least about 30 to 90 wt %; and the contacting conditions include a temperature of about 149° F. (about 65° C.) or less.

12. The method of claim 1, wherein the hydrocarbon fuel or fuel precursor feedstream comprises at least 5 wt % of a biocomponent feed.

13. The method of claim 6, wherein the hydrocarbon fuel or fuel precursor feedstream comprises at least 5 wt % of a biocomponent feed.

14. The method of claim 1, wherein the hydrocarbon fuel or fuel precursor feedstream remains as a liquid during the contacting with the sulfur sorbent material.

15. The method of claim 1, wherein at least a portion of the hydrocarbon fuel product is contacted with a reforming catalyst comprising at least one metal from Group VIII of the Periodic Table of Elements.

16. The method of claim 15, wherein the Group VIII metal is selected from platinum and palladium.

17. The method of claim 16, wherein the reforming catalyst further comprises at least one promoter metal from Group IIA, Group IVA, Group IB, Group VIB, and Group VIIB of the Periodic Table of Elements.

18. The method of claim 1, wherein the hydrocarbon fuel or fuel precursor feedstream is a product of a hydrocracking process; wherein the hydrocracking process comprises a hydrocracking catalyst which is a supported catalyst containing nickel, nickel-cobalt-molybdenum, cobalt-molybdenum, nickel-tungsten, or nickel-molybdenum components deposited thereon; and the hydrocracking process is operated at hydrocracking conditions which include a temperature from about 200° C. to about 450° C., a total pressure from about 5 barg (about 0.5 MPag) to about 300 barg (about 30 MPag), a hydrogen-containing treat gas ratio from about 100 scf/bbl (about 17 Nm³/m³) to about 5000 scf/bbl (about 840 Nm³/m³), and an LHSV from about 0.05 hr⁻¹ to about 10 hr⁻¹.
